(19) United States
(12) Patent Application Publication
(10) Pub. No.: US 2024/0032780 A1
(43) Pub. Date: Feb. 1, 2024

(54) MULTI-CAMERA ENDOSCOPES WITH MANEUVERABLE TIPS

(71) Applicant: 270 SURGICAL LTD., Netaya (IL)

(72) Inventors: Golan SALMAN, Atlit (IL); Avraham LEVY, Kfar Shmaryahu (IL); Amram AIZENFELD, Kibbutz Ramot Menashe (IL)

(21) Appl. No.: 18/268,754

(22) PCT Filed: Dec. 21, 2021
(86) PCT No.: PCT/IL2021/051517

§ 371 Date: Jun. 21, 2023

Related U.S. Application Data
(60) Provisional application No. 63/129,975, filed on Dec. 23, 2020.

(51) Int. Cl. A61B 1/00; A61B 1/05; A61B 1/06

(52) U.S. Cl. CPC A61B 1/00181; A61B 1/00177; A61B 1/05; A61B 1/00042; A61B 1/0625

(57) ABSTRACT

Provided herein are multi-camera endoscopes having a handle and an elongated member mounted on the handle and extending distally therefrom, the elongated member includes a proximally positioned shaft a distally positioned tip component; and a linking element affixed on a proximal section thereof to the shaft and on a distal section thereof to the tip component. Further provided are systems including the same and methods of use thereof in medical procedures.

### TECHNICAL FIELD

The present disclosure relates generally to multi-camera endoscopes with maneuverable tips.

### BACKGROUND

An endoscope is a medical device used to image an anatomical site (e.g. a body cavity, a hollow organ). Unlike some other medical imaging devices, the endoscope is inserted into the anatomical site (e.g. through small incisions made on the skin of the patient). An endoscope can be employed not only to inspect an anatomical site and e.g. organs therein (and diagnose a medical condition in the anatomical site) but also as a visual aid in surgical procedures. Medical procedures involving endoscopy include laparoscopy, arthroscopy, cystoscopy, ureteroscopy, and hysterectomy.

### SUMMARY

Aspects of the disclosure, according to some embodiments thereof, relate to multi-camera endoscopes with maneuverable tips. More specifically, but not exclusively, aspects of the disclosure, according to some embodiments thereof, relate to multi-camera endoscopes with maneuverable tips, which are configured to provide a user (e.g. a surgeon) with an indication of an orientation(s) of a side-camera(s) on the tip relative to the handle (which is held by the user) of the endoscope.

Manual maneuvering of an endoscope in small anatomical cavities, wherein, moreover, surgical devices may simultaneously be deployed, can prove challenging even for experienced users. Tip-maneuverable endoscopes aim to address this challenge by allowing imaging of an anatomical site at a plurality of orientations without requiring the user, who wields the endoscope, to manually tilt, rotate, or otherwise maneuver the endoscope. Nevertheless, manual manipulation of an endoscope, and, in particular, a multi-camera endoscope, naturally affords the user with information regarding the orientations of the cameras, and, in particular, the side-camera(s). More specifically, the handle of a (non-tip-maneuverable) multi-camera endoscope will typically indicate the direction, in which the side-camera(s) faces, via a visual and/or tactile marking. The present disclosure advantageously provides a tip-maneuverable multi-camera endoscope, which—similarly to a non-tip-maneuverable multi-camera endoscope—additionally provides a user with an indication regarding the orientation(s) of the side-camera(s), thereby facilitating identification of organs and organ parts during medical procedures involving endoscopy.

Thus, according to an aspect of some embodiments, there is provided a multi-camera endoscope. The endoscope includes a handle and an elongated member, which is mounted on the handle and extends distally therefrom (i.e. from the handle). The elongated member includes:
A proximally positioned shaft.
A distally positioned tip component, which may be elongated.
A linking element affixed on a proximal section thereof to the shaft and on a distal section thereof to the tip component.

The tip component includes a front camera and two opposite facing, or substantially opposite facing, side-cameras. The cameras are configured to jointly provide a field-of-view (FOV) of at least about 270 degrees. The endoscope houses at least one motor, and associated mechanical infrastructure, configured for controllable rotation of the tip component about a shaft longitudinal axis (i.e. a longitudinal axis of the shaft), thereby allowing to switch the endoscope between different configurations associated with different FOVs without moving or rotating the handle. The linking element is torsion-resistant, being thereby configured to maintain, when rotating the tip component about the shaft longitudinal axis, the side-cameras perpendicular, or substantially perpendicular, to a vertical axis. The vertical axis is perpendicular to the shaft longitudinal axis and defines, together with the shaft longitudinal axis, a vertical plane bisecting the endoscope.

According to some embodiments, a direction defined by the vertical axis is visually and/or tactually indicated on the handle, such that a user wielding the handle is thereby provided with information regarding orientations of the side-cameras.

According to some embodiments, the handle includes a user control interface on a top surface of the handle. The user control interface includes a plurality of buttons each pointing in a direction of the vertical axis.

According to some embodiments, the at least one motor includes one or more of an electric or electro-magnetic motor.

According to some embodiments, the endoscope further includes a coupling element. The coupling element includes a bent central part, a proximal arm, connected to the central part, and a distal arm connected to the (bent) central part, such that the distal arm is set at a fixed angle relative to the proximal arm. The proximal arm is mechanically coupled to the shaft and the distal arm is mechanically coupled to the tip component. The mechanical coupling between the shaft and the proximal arm is configured to prevent displacement of the proximal arm, and therefore the coupling element, relative to the shaft, but to allow for rotation of the coupling element about the shaft longitudinal axis independently of any rotation of the shaft about the shaft longitudinal axis. The mechanical coupling between the tip component and the distal arm is configured to prevent displacement of the distal arm, and therefore the coupling element, relative to the tip component, but to allow for rotation of the coupling element about a tip component longitudinal axis (i.e. a longitudinal axis of the tip component) independently of any rotation of the tip component about the tip component longitudinal axis. The mechanical coupling between the shaft and the proximal arm, and the mechanical coupling between the tip component and the distal arm, thus fix the tip component at the fixed angle relative to the shaft. The at least one motor, and associated mechanical infrastructure, are configured to controllably rotate the coupling element about the shaft longitudinal axis, thereby controllably rotating the tip component about the shaft longitudinal axis and switching the endoscope between the configurations.

According to some embodiments, the mechanical couplings between the shaft and the proximal arm, and between the tip component and the distal arm, additionally provide fluidic-sealing.

According to some embodiments, the fluidic sealing provided by the mechanical couplings between the shaft and the proximal arm, and between the tip component and the distal arm, are configured to withstand autoclave sterilization, and the endoscope is reusable. According to some such embodiments, the coupling element is metallic.

According to some embodiments, the coupling element may be made of a polymeric or ceramic material adapted for use in invasive medical procedures.

According to some embodiments, the endoscope may be configured for single-use (and disposable), or the elongated member may be detachably mounted on the handle and configured for single-use (and disposable).

According to some embodiments, the coupling element is integrally formed.

According to some embodiments, the endoscope further includes a coupling element. The coupling element includes a bent central part, a proximal arm, mechanically coupled to the (bent) central part, and a distal arm mechanically coupled to the central part, such that the distal arm is set at a fixed angle relative to the proximal arm. The proximal arm is connected to the shaft and the distal arm is connected to the tip component, thereby fixing the tip component at the fixed angle relative to the shaft. The mechanical coupling between the proximal arm and the central part is configured to prevent displacement of the proximal arm, and therefore the shaft, relative to the central part, but to allow for rotation of the central part about the shaft longitudinal axis. The mechanical coupling between the distal arm and the central part is configured to prevent displacement of the distal arm, and therefore the tip component, relative to the central part, but to allow for rotation of the distal arm and the tip component about a tip component longitudinal axis. The at least one motor, and associated mechanical infrastructure, are configured to controllably rotate the central part about the shaft longitudinal axis, thereby controllably rotating the distal arm and the tip component about the shaft longitudinal axis and switching the endoscope between the configurations.

According to some embodiments, the mechanical couplings between the proximal arm and the central part, and between the distal arm and the central part, additionally provide fluidic-sealing, such as to withstand autoclave sterilization.

According to some embodiments, the fixed angle is between about 10 degrees and about 30 degrees.

According to some embodiments, the coupling element is hollow and the linking element extends therethrough.

According to some embodiments, the linking element is a torsion-resistant wire, cable, tube, or bellows.

According to some embodiments, the linking element is a hollow bellows connected on a proximal end thereof to a distal end of the shaft and on a distal end thereof to a proximal end of the tip component. The coupling element extends through the bellows.

According to some embodiments, the bellows is made of a material configured to withstand autoclave sterilization. Further, the bellows connection to the shaft and the bellows connection to the tip component are fluidly-sealed, such as to withstand autoclave sterilization.

According to some embodiments, the at least one motor, and the associated mechanical infrastructure, are further configured to controllably modify an inclination angle defined between the shaft longitudinal axis and a tip component longitudinal axis.

According to some embodiments, the inclination angle may be controllably set at any of a plurality of angles in a range of angles from 0 degrees to about 30 degrees.

According to some embodiments, the plurality of angles constitutes a continuous range of angles.

According to some embodiments, the linking element is a bellows connected on a proximal end thereof to a distal end of the shaft and on a distal end thereof to a proximal end of the tip component.

According to some embodiments, the bellows is made of a material configured to withstand autoclave sterilization.

According to some embodiments, the bellows is made of a metallic material. According to some such embodiments, the bellows is made of, or includes stainless steel.

According to some embodiments, the bellows connection to the shaft and the bellows connection to the tip component are fluidly-sealed.

According to some embodiments, the bellows connections to the shaft and the tip component are configured to withstand autoclave sterilization, and the endoscope is reusable.

According to some embodiments, the bellows is made of a polymeric, ceramic, or metallic material adapted for use in invasive medical procedures.

According to some embodiments, the endoscope may be configured for single-use (and disposable), or the elongated member may be detachably mounted on the handle and configured for single-use (and disposable).

According to some embodiments, the side-cameras are not positioned back-to-back.

According to some embodiments, each of the cameras includes a CMOS (complementary metal-oxide semiconductor) image sensor and/or a CCD (charge-coupled device) image sensor.

According to some embodiments, the tip component further includes a plurality of illumination modules configured to jointly illuminate the FOV of the cameras.

According to some embodiments, each of the illumination modules includes one or more light-emitting diodes (LEDs).

According to some embodiments, there is provided a system which includes the endoscope as disclosed herein and a main control unit configured to control operation of the endoscope.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the Figures:

FIG. 1A presents a schematic side-view of an endoscope including an elongated member mounted on a handle, the elongated member including a shaft and a distal tip component disposed at an angle relative to the shaft, the distal tip component being mechanically coupled to the shaft via a coupling element, which is configured for switching the endoscope between different configurations associated with different combined field-of-views of cameras on the distal tip component, according to some embodiments;

FIG. 1B presents a schematic perspective view of the endoscope of FIG. 1A, according to some embodiments;

FIG. 2 schematically depicts a distal portion of the endoscope FIG. 1A and an associated combined field-of-view provided by cameras; a top surface of the distal tip component is removed to reveal internal components thereof, according to some embodiments;

FIG. 3A-3C present schematic views of a distal portion of the endoscope of FIG. 1A in three respective configurations, according to some embodiments;

FIGS. 4A-4C schematically depict three configurations of the endoscope of FIG. 1A, respectively, according to some embodiments;

FIGS. 5A and 5B present schematic top-views of the endoscope of FIG. 1A in two configurations, respectively, and associated combined field-of-views provided by cameras, according to some embodiments;

FIGS. 6A-6C present schematic views of the coupling element of the endoscope of FIG. 1A, according to some embodiments;

FIG. 7 is a block diagram relating functionality of some parts and components of the endoscope of FIG. 1, according to some embodiments;

FIGS. 8A and 8B present schematic side-views of an endoscope in two respective configurations, the endoscope including an elongated member mounted on a handle, the elongated member including a shaft and a distal tip component positioned at an angle relative to the shaft, the distal component being mechanically coupled to the shaft via an elongated neck element, according to some embodiments;

FIGS. 9A-9B and 10 present schematic views of a distal portion of the endoscope of FIG. 8A, according to some embodiments; and

FIG. 11 presents a schematic view of a distal portion of the endoscope of FIG. 8A, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 90% and 110% of the given value. In such embodiments, for example, the statement “the length of the element is equal to about 1 millimeter” is equivalent to the statement “the length of the element is between 0.90 millimeters and 1.10 millimeters”. According to some embodiments, “about” may specify the value of a parameter to be between 95% and 105% of the given value. According to some embodiments, “about” may specify the value of a parameter to be between 91% and 101% of the given value.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol └ may be used to represent an axis pointing “out of the page”, while the symbol ⊗ may be used to represent an axis pointing “into the page”.

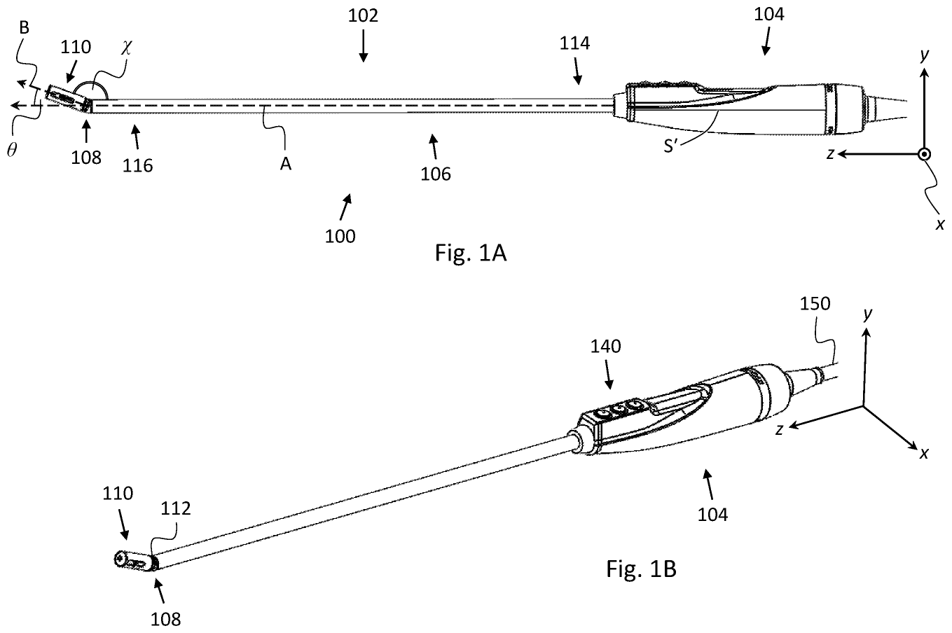

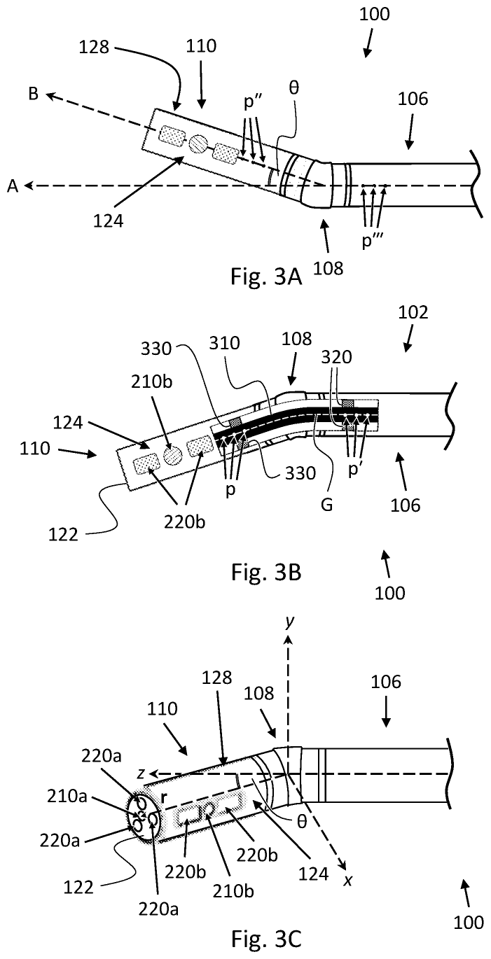

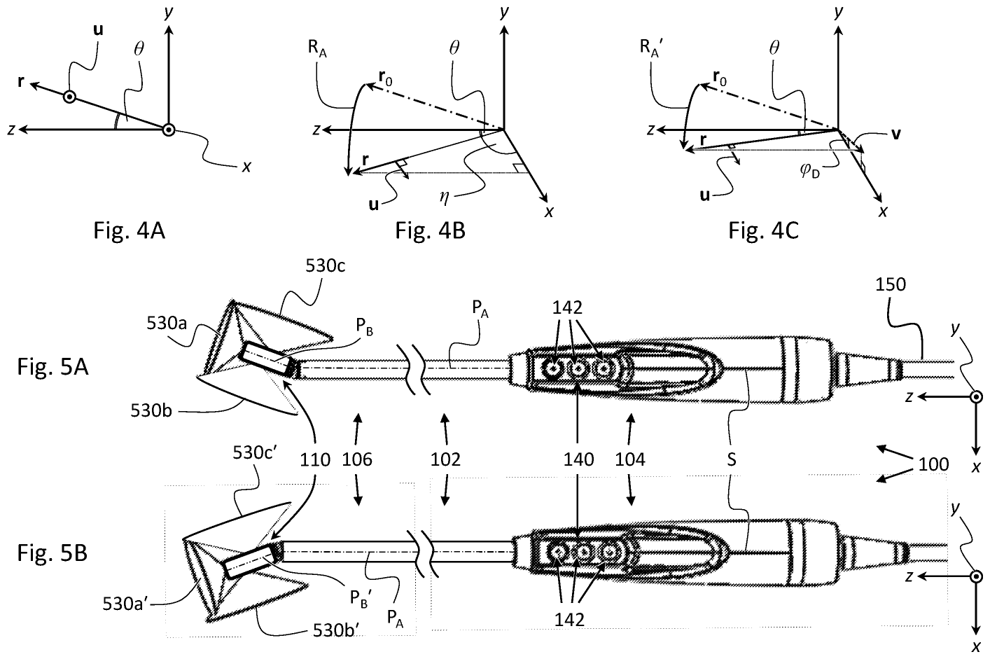

FIGS. 1A and 1B schematically depict an endoscope 100, according to some embodiments. Endoscope 100 includes an elongated member 102, configured to be inserted into an anatomical site (e.g. an anatomical cavity), and a handle 104, configured to be held by a user (e.g. a surgeon) of endoscope 100 and to facilitate guiding and manipulation of elongated member 102 (particularly the distal portion thereof) within the anatomical site. Elongated member 102 includes a shaft 106, a coupling element 108, and a (distal) tip component 110. Coupling element 108, the function of which is described below, is mounted on a shaft distal end 112 (i.e. the distal end of shaft 106). Tip component 110 is mounted on coupling element 108, such as to be set at a fixed angle θ (indicated also in FIGS. 3A, 3C, and 4A-4C) relative to shaft 106.

More precisely, the angle θ (also referred to as the “inclination angle”) extends between a longitudinal axis A of shaft 106 and a longitudinal axis B of tip component 110. The longitudinal axis A centrally extends through shaft 106 along the length thereof. The longitudinal axis B centrally extends through tip component 110 along the length thereof. An angle χ, which is spanned between tip component 110 and shaft 106, and which is supplementary to the angle θ (i.e. χ=180°−θ), is indicated in FIG. 1A. A shaft proximal portion 114 is connected to, or detachably connected to, handle 104, thereby mounting elongated member 102 on handle 104.

According to some embodiments, the inclination angle θ is between about 1 degree and about 45 degrees. According to some embodiments, the inclination angle θ is between about 5 degrees and about 40 degrees. According to some embodiments, the inclination angle θ is between about 10 degrees and about 30 degrees. According to some embodiments, the inclination angle θ is between about 15 degrees and about 30 degrees. According to some embodiments, the inclination angle θ is between about 15 degrees and about 22 degrees.

According to some embodiments, each of shaft 106 and tip component 110 may have a round or substantially round transverse cross-section. According to some embodiments, tip component 110 may be of a greater diameter than shaft 106 or at least a shaft distal portion 116 (which includes shaft distal end 112), as described in PCT application publication No. WO2019035118 to A. Levy et al., which is incorporated herein by reference in its entirety. According to some such embodiments, a proximal portion of tip component 110 may be tapered (i.e. grow narrower in the proximal direction).

According to some embodiments, elongated member 102 may measure between about 100 millimeters and about 500 millimeters in length. According to some embodiments, each of shaft 106 and tip component 110 may have a diameter measuring between about 2 millimeters and about 15 millimeters. According to some embodiments, tip component 110 may measure between about 6 millimeter and about 25 millimeters in length.

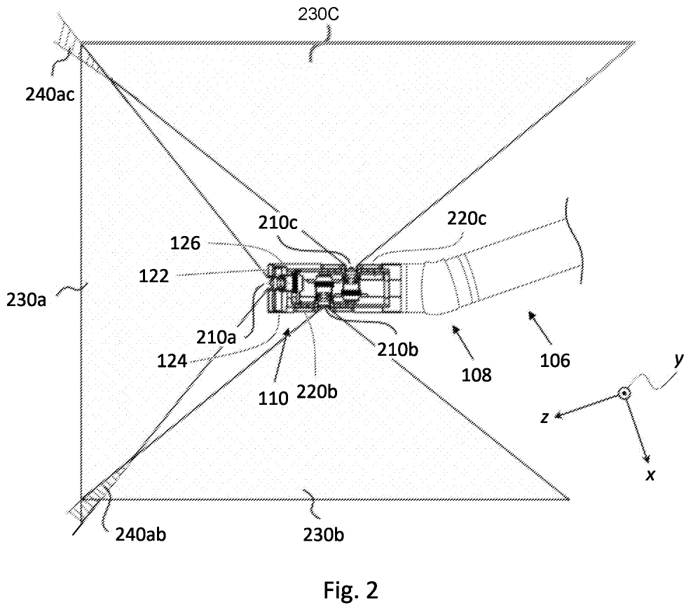

Tip component 110 includes a front surface 122, a first side-surface 124, and a second side-surface 126 (not visible in FIGS. 1A and 1B; indicated in FIG. 2) positioned oppositely to first side-surface 124. A top surface 128 of tip component 110 is indicated in FIGS. 3A and 3C. Top surface 128 extends between first side-surface 124 and second side-surface 126.

Tip component 110 may include a plurality of cameras: a front camera and at least one side-camera. According to some embodiments, and as depicted in FIG. 2, tip component 110 includes three cameras. FIG. 2 presents a top view of tip component 110, wherein top surface 128 has been removed to reveal internal components of tip component 110, according to some embodiments. Tip component 110 is shown including three cameras 210 and associated illumination modules 220 (not all of which are numbered). Each of cameras 210 includes a lens assembly and an image sensor (not numbered). According to some embodiments, each of the image sensors is a CMOS (complementary metal-oxide semiconductor) image sensor, but it will be understood that other options are possible. In particular, according to some alternative embodiments, one or more of the image sensors may be a CCD (charge-coupled device) image sensor.

According to some embodiments, cameras 210 include a front camera 210a, a first side-camera 210b, and a second side-camera 210c. Front camera 210a is positioned within tip component 110, with a front lens assembly (not numbered) of front camera 210a being positioned adjacently to, or on, front surface 122. Front camera 210a further includes front image sensor (not numbered). First side-camera 210b is positioned within tip component 110, with a first side-lens assembly (not numbered) of first side-camera 210b being positioned adjacently to, or on, first side-surface 124. First side-camera 210b further includes a first side-image sensor (not numbered). Second side-camera 210c is positioned within tip component 110, with a second side-lens assembly (not numbered) of second side-camera 210c being positioned adjacently to, or on, second side-surface 126. Second side-camera 210c further includes a second side-image sensor (not numbered).

According to some embodiments, and as depicted in FIG. 2, first side-camera 210b and second side-camera 210c are not positioned back-to-back. According to some embodiments, the distance between the center-point of first side-camera 210b (e.g. the center of the lens assembly of first side-camera 210b) and front surface 122 is between about 5 millimeters to about 20 millimeters and the distance between the center-point of first side-camera 210b and the center-point of second side-camera 210c may be up to about 10 millimeters.

Also indicated in FIG. 2 is a three-dimensional cartesian coordinate system and a combined (joint) horizontal field-of-view (FOV) provided by cameras 210, according to some embodiments. The combined horizonal FOV is formed by a front horizonal FOV 230a, a first side-horizonal FOV 230b, and a second side-horizonal FOV 230c of front camera 210a, first side-camera 210b, and second side-camera 210c, respectively. Each of the horizonal FOVs 230a, 230b, and 230c lies on the zx-plane. Front horizonal FOV 230a is positioned between side horizonal FOVs 230b and 230c and overlaps with each. A first overlap area 240ab corresponds to an area whereon horizonal FOVs 230a and 230b overlap. In other words, first overlap area 240ab is defined by the intersection of the zx-plane with the overlap region (volume) of the FOVs of front camera 210a and first side-camera 210b. Similarly, a second overlap area 240ac corresponds to an area whereon horizonal FOVs 230a and 230c overlap.

According to some embodiments, the combined horizonal FOV spans between about 220 degrees to about 270 degrees, between about 240 degrees to about 300 degrees, or between about 240 degrees to about 340 degrees. Each possibility corresponds to separate embodiments. According to some embodiments, the combined horizonal FOV spans at least about 270 degrees. According to some embodiments, for example, each of horizonal FOVs 230a, 230b, and 230c may measure between about 85 degrees to about 120 degrees, between about 90 degrees to about 110 degrees, or between about 95 degrees to about 120 degrees. Each possibility corresponds to separate embodiments.

According to some embodiments, front camera 210a may face in the direction, or substantially in the direction, of the longitudinal axis B (shown in FIGS. 1A and 3A) of tip component 110. According to some embodiments, first side-camera 210b may face transversely, or substantially transversely. More specifically, first side-camera 210b may point in parallel, or substantially in parallel to the zx-plane, and perpendicularly, or substantially perpendicularly to the longitudinal axis B. According to some embodiments, second side-camera 210c may face in an opposite, or substantially opposite, direction to first side-camera 210b. According to some embodiments, the two side-cameras may be positioned such that they are not back-to-back.

According to some embodiments, front camera 210a may be offset (i.e. laterally displaced) relative to the longitudinal axis B. According to some embodiments, and as depicted in FIG. 2, the distance between second side-camera 210c and front surface 122 is greater than the distance between first side-camera 210b and front surface 122. According to some alternative embodiments, the distance between second side-camera 210c and front surface 122 is smaller than the distance between first side-camera 210b and front surface 122.

According to some embodiments, each of illumination modules 220 is associated with a respective camera from cameras 210. In particular, according to some embodiments, more than one illumination module may be associated with each of cameras 210. The illumination modules associated with a camera are configured to illuminate the FOV of the camera. For example, first side-illumination modules 220b (numbered in both of FIGS. 3B and 3C), which are positioned proximally and distally to first side-camera 210b, respectively, are configured to illuminate the FOV of first side-camera 210b. Similarly, front illumination modules 220a (numbered in FIG. 3C) are configured to illuminate the FOV of front camera 210a. According to some embodiments, each of the illumination modules includes one or more light emitting diodes (LEDs).

Referring again to FIGS. 1A and 1B, handle 104 may include a user control interface 140 configured to allow a user to control endoscope 100 functions. In particular, user control interface 140 may be functionally associated with cameras 210 and illumination modules 220. According to some embodiments, user control interface 140 may allow, for example, to control zoom, focus, record/stop recording, and/or freeze frame functions of cameras 210 and/or to adjust the intensity of light provided by illumination modules 220 collectively and/or individually. User control interface 140 may include one or more buttons, knobs, switches, a touch panel, and/or the like. According to some embodiments, and as depicted in FIGS. 1A, 1B, 5A and 5B, user control interface 140 may include one or more buttons, which point vertically, that is, in the direction defined by the y-axis. According to some embodiments, user control interface 140 includes a plurality of buttons 142, which may be linearly arranged along a length of handle 104. In other words, buttons 142 are linearly arranged in parallel to the z-axis. According to some such embodiments, buttons 142 are arranged along a topmost longitudinal strip S (indicated in FIGS. 5A and 5B) on the surface of handle 104 and face vertically.

According to some embodiments, endoscope 100 may be (i) directly maneuvered by a user through the manipulation of handle 104, as well as (ii) indirectly maneuvered, via robotics, e.g. using a robotic arm or other suitable gripping means configured to allow manipulation of handle 104.

According to some embodiments, endoscope 100 is functionally associated, or associable, with a main control unit (not shown). The main control unit may include electronic circuitry (e.g. one or more processors and memory components) configured to process (digital data) from cameras 210, such as to display images and video(s) (captured by cameras 210) on a monitor. In particular, the processing circuitry may be configured to process the digital data received from each of cameras 210, such as to produce therefrom a combined video file/stream providing a continuous and consistent (seamless) panoramic view of an anatomical site wherein endoscope 100 is inserted.

According to some embodiments, endoscope 100 may be functionally associated with the main control unit via a utility cable 150. According to some such embodiments, utility cable 150 may also serve as a power cable. That is, in such embodiments, utility cable 150 may further provide electricity to power endoscope 100 operation. According to some alternative embodiments, the main control unit may be functionally associated with endoscope 100 through wireless communication.

FIGS. 3A-3C schematically depict endoscope 100 (not shown in full) in three respective configurations between which endoscope 100 is switchable, according to some embodiments. To facilitate the description, a cartesian coordinate system is introduced, such that the longitudinal axis A of shaft 106 coincides with the z-axis. The coordinate system is shown superimposed on endoscope 100 in FIG. 3C. Coupling element 108 is positioned at the origin of the coordinate system. (In this regard, it is noted that the coordinate system is identical to the coordinate systems indicated in FIGS. 1A-2, except that the origin has been translated to coincide with a center-point of coupling element 108.) The position of a center-point of front surface 122 is given by a vector r=x\hat{x}+yŷ)+z\hat{z}=r\hat{r}, wherein r is the magnitude of r. The longitudinal axis B of tip component 110 thus coincides with the vector r and the angle between the vector r and the z-axis is given by the inclination angle θ.

It is noted that in each of FIGS. 3A-3C, first side-camera 210b is shown pointing in parallel to the zx-plane. According to some embodiments, and as further elaborated on below, endoscope 100 is switchable across a range of configurations in each of which first side-camera 210b and second side-camera 210c each points in parallel to the zx-plane.

Referring also to FIGS. 4A-4C, FIGS. 4A-4C provide a schematical, geometrical illustration of configurations between which endoscope 100 is switchable, according to some embodiments. FIGS. 4A and 4B schematically illustrate the configurations of FIGS. 3A and 3C, respectively. FIG. 4C schematically illustrates an additional configuration of endoscope 100, which constitutes a mid-configuration between the configurations of FIGS. 4A and 4B. In each of FIGS. 4A-4C the vector r is depicted together with the x, y, and z axes of the cartesian coordinate system defined above (and shown in FIG. 3C). In terms of spherical coordinates, the vector r is defined by the three coordinates (r, θ, φ). Here θ is the polar angle (the spherical coordinates are selected such that the polar angle coincides with the inclination angle defined above). φ is the azimuthal angle, which is defined as the angle between the x-axis and the projection of the vector r on the xy-plane. More specifically, r=r(sin(θ) cos(φ) \hat{x}+sin(θ) sin(φ) ŷ+cos(θ) \hat{z}).

In FIG. 4A the vector r is positioned on the yz-plane in the positive quadrant thereof (i.e. the quadrant wherein both y>0 and z>0). The azimuthal angle φ therefore equals 90 degrees. In FIG. 4B the vector r is positioned on the zx-plane in the positive quadrant thereof. The azimuthal angle φ therefore equals 0 degrees. In FIG. 4C the vector r is positioned in the positive octant (i.e. the octant wherein x>0, y>0, and z>0). The azimuthal angle φ equals φ_{D}, wherein 0°<φ_{D}<90°. Also indicated in FIG. 4C is a vector v indicating the projection of the vector r on the xy-plane.

In each of FIGS. 4A-4C a (respective unit) vector u—pointing in parallel to the zx-plane and perpendicularly to the vector r (as well as the y-axis)—indicates the direction along which first side-camera 210b faces. In FIG. 4A, the vector u points in the direction of the positive x-axis. In FIG. 4B, the vector u is positioned on the zx-plane, and points along the direction defined by the unit vector cos(θ)\hat{x}−sin(θ) \hat{z}. According to some embodiments, the configuration of FIG. 4B may be obtained from that of FIG. 4A by a 90 degrees clockwise rotation of tip component 110 (i.e. the vector r) about the z-axis (or, what is the same thing, the longitudinal axis A), as indicated by a curved arrow R_{A}, and as elaborated on below. The position and orientation of the vector r prior to the rotation thereof is indicated by a vector r₀ (indicated by a dashed-dotted arrow). Also indicated is an angle η=90°−θ spanned between the vector r and the x-axis. In FIG. 4C, the vector u is positioned above the zx-plane, and points in the direction defined by the vector cos(μ) \hat{x}−sin(μ) \hat{z}, wherein 0°<μ<θ. According to some embodiments, the configuration of FIG. 4C may be obtained from that of FIG. 4A by a (90°−φ_{D}) clockwise rotation of tip component 110 (i.e. the vector r) about the z-axis, as indicated by a curved arrow R_{A}′.

According to some embodiments, the different configurations of endoscope 100 may be characterized by the same value of the inclination angle θ, but may differ from one another in the value of the azimuthal angle φ. According to some such embodiments, endoscope 100 is switchable across a continuous range of azimuthal angles. According to some such embodiments, the range may allow for full, or substantially full, rotation of tip component 110 about the z-axis (that is, the longitudinal axis A of shaft 106), such that side-cameras 210b and 210c face as described above (specifically, perpendicularly, or substantially perpendicularly, to they-axis). According to some embodiments, tip component 110 may be rotated both clockwise and counter-clockwise (i.e. anti-clockwise) about the z-axis.

More specifically, a side-camera which faces perpendicularly to the respective side-surface of tip component 110 (and therefore perpendicularly to the longitudinal axis B of tip component) will always remain perpendicular to the y-axis as endoscope 100 is switched between configurations. In contrast, a side-camera, which—when endoscope 100 is in a configuration wherein tip component 110 (and the rest of elongated member 102) is bisected by the zx-plane—faces in a direction along the zx-plane that is slightly tilted with respect to the x-axis, in any other configuration of endoscope 100 will only substantially point perpendicularly to the y-axis.

Each of FIGS. 5A and 5B schematically depicts endoscope 100 in respective configurations, according to some embodiments. Corresponding combined FOVs in each of the configurations are also depicted. A straight line PA serves to indicate a first vertical plane, which is parallel to the yz-plane, and which vertically bisects shaft 106 (and therefore includes the longitudinal axis A). A straight line PB in FIG. 5A serves to indicate a second vertical plane, which is parallel to the y-axis, and which bisects tip component 110 (and therefore includes the longitudinal axis B). Hence, the angle between the first vertical plane and the second vertical plane (i.e. between the respective normal vectors thereto) is equal to the azimuthal angle characterizing the configuration of endoscope 100 in FIG. 5A. Similarly, a straight line P_{B}′ in FIG. 5B serves to indicate a third vertical plane, which is parallel to the y-axis, and which bisects tip component 110 (and therefore includes the longitudinal axis B). The angle between the first vertical plane and the third vertical plane is equal to the azimuthal angle characterizing the configuration of endoscope 100 in FIG. 5B.

Referring to FIG. 5A, indicated are a front FOV 530a of front camera 210a, a first-side FOV 530b of first side-camera 210b, and a second-side FOV 530c of second-side camera 210c, according to some embodiments. Referring to FIG. 5B, indicated are a front FOV 530a′ of front camera 210a, a first-side FOV 530b′ of first side-camera 210b, and a second-side FOV 530c′ of second-side camera 210c, according to some embodiments.

As described above, according to some embodiments, user control interface 140 includes buttons 142, which may be linearly arranged along a topmost longitudinal strip on the surface of handle 104. It is noted that in such embodiments, buttons 142 point in the direction of the positive y-axis. Since side-cameras 210b and 210c always, or substantially always, point perpendicularly, or substantially perpendicularly, to they-axis (or what amounts to the same thing, in parallel, or substantially in parallel, to the zx-plane), buttons 142 provide a user, who wields endoscope 100, with a visual cue regarding directions (e.g. opposite directions or substantially opposite directions) along which each of first side-camera 210b and second side-camera 210c, respectively, faces. Additionally, or alternatively (e.g. in embodiments, wherein user control interface 140 of itself does not provide a visual cue), handle 104 may include a marking (visual and/or tactile) providing an indication as to directions in which each of side-cameras 210b and 210c, respectively, faces. The marking may be, for example, a distinctly colored strip along a length of handle 104 on the top thereof, e.g. in embodiments wherein the longitudinal strip S is marked. The marking may be, for example, a linear groove (straight depression) or a linear ridge (straight projection) along a length of handle 104 on the top thereof, e.g. in embodiments wherein the longitudinal strip S is slightly raised or depressed, respectively. The marking may be, for example, a plurality of linearly arranged projections (e.g. rounded projections) and/or notches (e.g. rounded notches) along the longitudinal strip S. The projections and/or grooves may be distinctly colored. Further, markings such as described above may additionally, or alternatively, be disposed in a different manner along the surface of handle 104. For example, according to some embodiments, the marking may be a linear groove or a linear ridge along an outermost longitudinal strip S′ (indicated in FIG. 1A) on handle 104 and/or on the opposite side of handle 104.

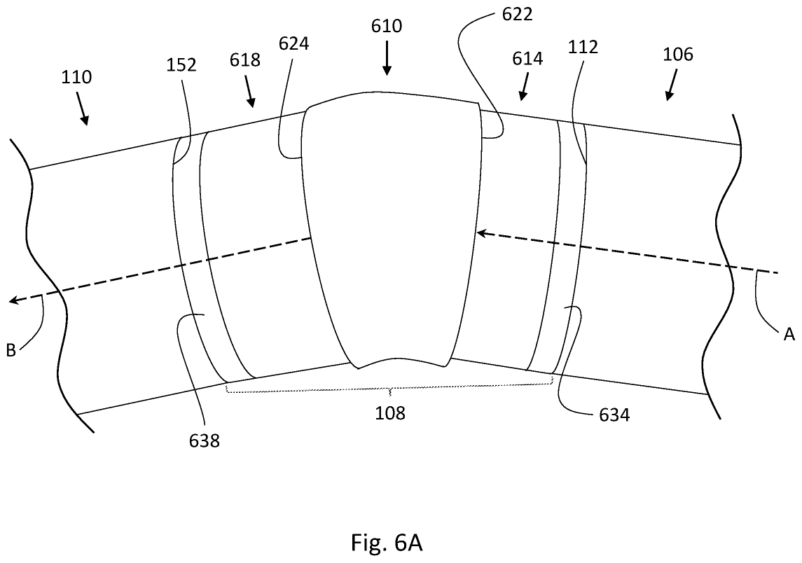

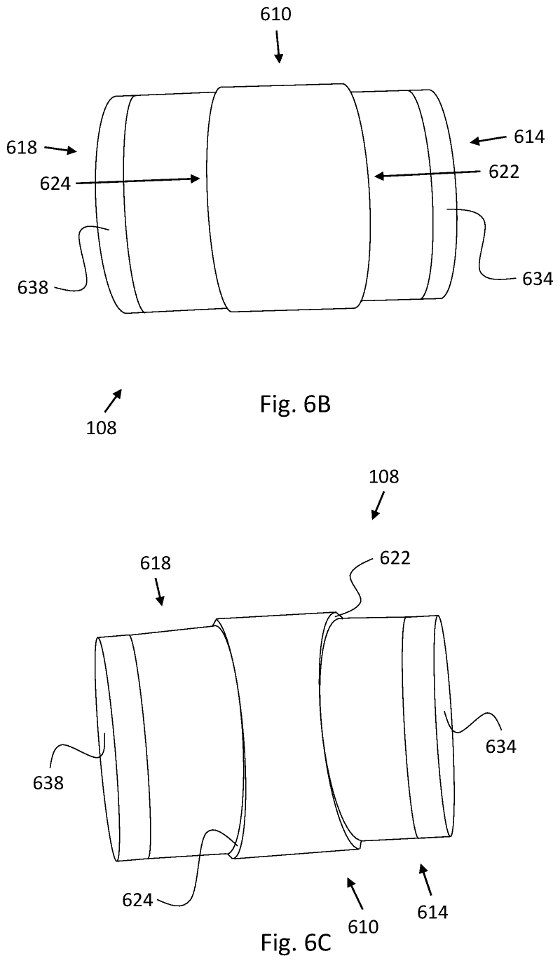

Mechanisms whereby the above-described switching between configurations may be implemented are described next. FIGS. 6A-6C schematically depict coupling element 108, according to some embodiments. FIG. 6A provides a schematic side-view of coupling element 108, according to some embodiments. FIG. 6B provides a schematic top-view of coupling element 108, according to some embodiments. FIG. 6C provides a schematic bottom-view of coupling element 108, according to some embodiments.

Referring to FIG. 6A, according to some embodiments, coupling element 108 includes a (bent) central part 610, a proximal arm 614, and a distal arm 618. Proximal arm 614 is connected to central part 610 on a central part proximal end portion 622 (i.e. a proximal end portion of central part 610). Distal arm 618 is connected to central part 610 on a central part distal end portion 624 (i.e. a distal end portion of central part 610).

According to some embodiments, coupling element 108 is integrally formed. According to some such embodiments, coupling element 108 may be fabricated by casting or molding, by machining of a single piece of material, and/or by additive manufacturing.

According to some embodiments, for example, embodiments wherein endoscope 100 is reusable, coupling element 108 may be made of a material having resistance to repeated steam autoclaving without loss of dimensional stability or change in physical characteristics thereof, and which is, moreover, adapted for use in invasive medical procedures. As non-limiting examples, coupling element 108 may be made of a metallic material, e.g. stainless steel, or a polymeric material, such as polyphenylsulfone (which is configured to withstand autoclave sterilization). According to some alternative embodiments, wherein endoscope 100 is a single-use disposable device, or wherein elongated member 102 is detachably mounted on handle 104 and configured for single-use, coupling element 108 may be made of any material adapted for use in invasive medical procedures, such as a suitable plastic, ceramic, and/or metallic material.

According to some embodiments, proximal arm 614 may be directly mechanically coupled, on a proximal arm external end portion 634 (i.e. a proximal end portion of proximal arm 614), to shaft distal end 112, such that proximal 614 is aligned with shaft 106 along the longitudinal axis A of shaft 106. The mechanical coupling may be configured to prevent axial displacement (i.e. along the longitudinal axis A) of proximal arm 614 relative to shaft 106, but to allow proximal arm 614 rotation, and therefore coupling element 108 rotation, about the longitudinal axis A. The mechanical coupling may further be configured to provide fluidic-sealing (e.g. by means of a tightening mechanism and/or use of rubber or stainless steel O-rings), such as to prevent debris and fluids from entering into endoscope 100 (via the interface between proximal arm external end portion 634 and shaft distal end 112) during use, and allow endoscope 100 to withstand autoclave sterilization. According to some embodiments, proximal arm external end portion 634 may form an inner extension of shaft 106, extending therefrom in the distal direction and centered about the longitudinal axis A of shaft 106.

Similarly, according to some embodiments, distal arm 618 may be directly mechanically coupled, on a distal arm external end portion 638 (i.e. a distal end portion of distal arm 618), to a proximal end 152 of tip component 110, such that distal arm 618 is aligned with tip component 110 along the longitudinal axis B of tip component 110. The mechanical coupling may be configured to prevent axial displacement (i.e. along the longitudinal axis B) of distal arm 618 relative to tip component 110, but to allow tip component 110 rotation about the longitudinal axis B, which is not accompanied by a rotation of distal arm 618 about the longitudinal axis B. The mechanical coupling may further be configured to provide fluidic-sealing (e.g. by means of a tightening mechanism and/or use of rubber or stainless steel O-rings), such as to prevent debris and fluids from entering into endoscope 100 (via the interface between distal arm external end portion 638 and the proximal end of tip component 110) during use, and allow endoscope 100 to withstand autoclave sterilization. According to some embodiments, distal arm external end portion 638 may form an inner extension of tip component 110, extending therefrom in the proximal direction and centered about the longitudinal axis B of tip component 110.

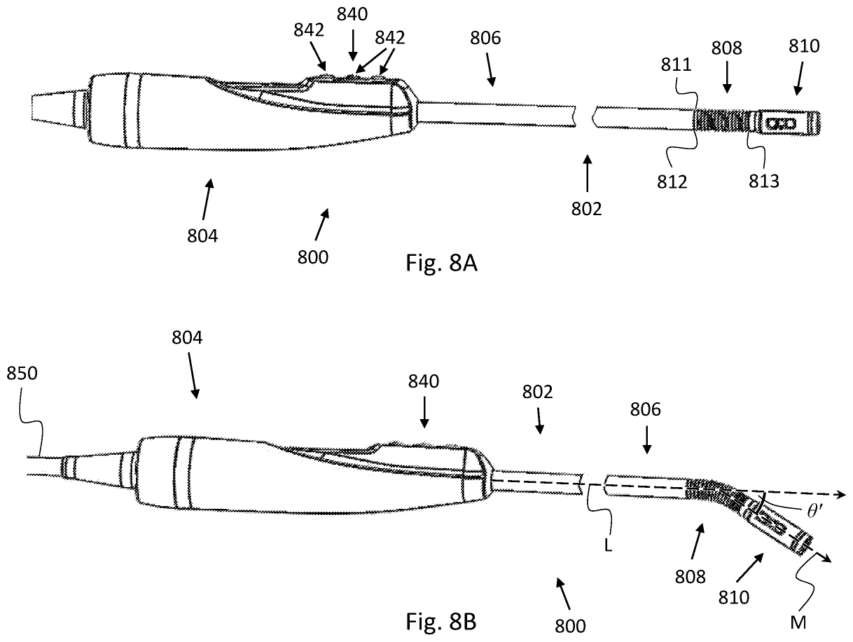

Referring again to FIG. 3B, elongated member 102 is shown with a surface-portion thereof cut away to reveal some internal components of elongated member 102, according to some embodiments. Depicted is an elongated element 310 (also referred to as “linking element”). Elongated element 310 extends distally from shaft 106 to tip component 110, at least along shaft distal portion 116 and along the longitudinal axis A, through coupling element 108, and along at least a proximal portion of tip component 110 and along the longitudinal axis B. Elongated element 310 may be flexible, at least along a segment thereof extending through coupling element 108. Elongated element 310 is highly torsion-resistant in the sense of not allowing, or effectively not allowing, for elongated element 310 to be wound about itself. According to some embodiments, elongated element 310 may be a highly torsion-resistant solid piece of wire (e.g. a metallic or plastic wire) or may be a torsion-resistant cable (e.g. formed of braided wires and/or formed similarly to a torsion-resistant rope). According to some embodiments, elongated element 310 may be a hollow tube or a hollow and flexible bellows (similar to the bellows of FIGS. 8A-10). According to some such embodiments, signal, electricity, and/or power transferring means/elements—such as, but not limited to, cables, electrical wires, optical fibers, and/or printed circuit boards (PCBs; which may be flexible, rigid, flex-rigid, or any combination thereof), coupled to electronic components (e.g. cameras 210 and/or illumination modules 220) in tip component 110—may extend through the tube. According to some embodiments, elongated element 310 may be a solid, in the sense of not being hollow, and flexible bellows (in which case, signal, electricity, and/or power transferring means/elements may extend through elongated member 102 adjacently to the bellows).

Also shown in FIG. 3B are connecting elements 320, positioned within shaft 106, and connecting elements 330, positioned within tip component 110. Connecting elements 320 affix elongated element 310 to shaft 106. In particular, connecting elements 320 prevent relative rotational motion between shaft 106 and the portion of elongated element 310 disposed along shaft 106. According to some embodiments, each of connecting elements 320 may form a rung extending laterally (i.e. perpendicularly to the longitudinal axis A), or substantially laterally, from elongated element 310 to an inner wall (not numbered) of shaft 106. Each of the rungs may be glued, fused, and/or anchored, or otherwise secured on a first end thereof to elongated element 310 and on a second end thereof to the inner wall of shaft 106. According to some embodiments, the rungs may be rigid.

Similarly, connecting elements 330 affix elongated element 310 to tip component 110. In particular, connecting elements 330 prevent relative rotational motion between tip component 110 and the portion of elongated element 310 disposed along tip component 110. According to some embodiments, each of connecting elements 330 may form a rung extending laterally (i.e. perpendicularly to the longitudinal axis B), or substantially laterally, from elongated element 310 to an inner wall (not numbered) of tip component 110. Each of the rungs may be glued, fused, and/or anchored, or otherwise secured on a first end thereof to elongated element 310 and on a second end thereof to the inner wall of tip component 110. According to some embodiments, the rungs may be rigid.

According to some embodiments, elongated element 310 may be, or may include, a torsion-resistant strip. The strip may be glued, or otherwise affixed, on a proximal section thereof, to an inner wall of shaft 106, and on a distal section thereof, to an inner wall of tip component 110.

It is noted that elongated element 310 mechanically couples shaft 106 to tip component 110, independently of the mechanical coupling there between provided by coupling element 108.

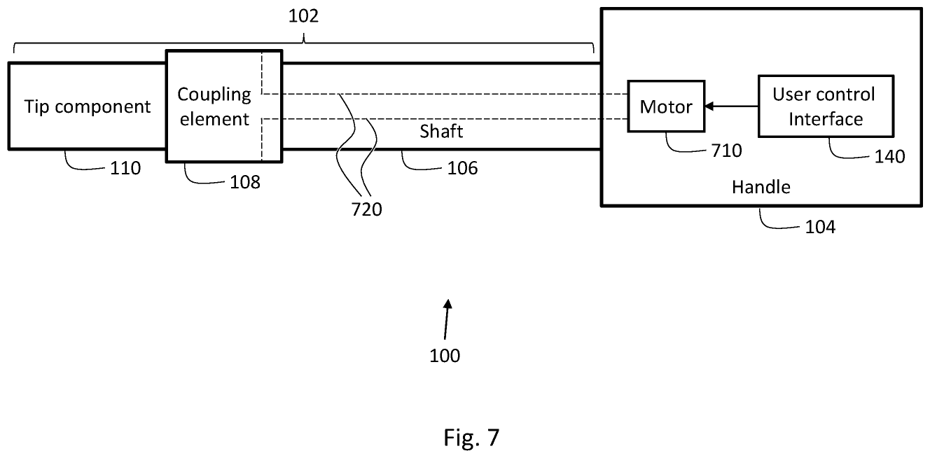

Referring again to FIG. 6A, to switch between configurations, coupling element 108 is rotated about the longitudinal axis A of shaft 106. The rotation may be implemented by a motor (shown in FIG. 7) included in endoscope 100 (e.g. within handle 104), which is mechanically associated with coupling element 108.

As explained above, proximal arm 614 of coupling element 108 is not rotationally coupled to shaft 106 in the sense that rotation of coupling element 108 about the longitudinal axis A does not force rotation of shaft 106. Similarly, distal arm 618 of coupling element 108 is not rotationally coupled to tip component 110 in the sense that rotation of coupling element 108 about the longitudinal axis B does not force rotation of tip component 110. Nevertheless, the mechanical coupling between distal arm 618 and tip component 110 prevents relative motion there between along the longitudinal axis B and maintains distal arm 618 and tip component 110 aligned (in the sense that neither may be laterally displaced relative to the longitudinal axis B). Consequently, rotation of coupling element 108 about the longitudinal axis A (of shaft 106) induces a rotation of tip component 110 about the longitudinal axis A, with distal arm 618 (or more precisely, a proximal end portion thereof) serving as the pivot point of the rotation of tip component 110.

Since elongated element 310 is torsion-resistant (i.e. cannot be wound about the longitudinal axis thereof), as tip component 110 is rotated about the longitudinal axis A (e.g. due to coupling element 108 rotation about the longitudinal axis A), groups of locations (pointwise sites) along the length of elongated element 310, which are linearly arranged there along—such as a group of locations on elongated element 310 within shaft 106, indicated by points p′, and such as a group of locations on elongated element 310 within tip component 110, indicated by points p, in FIG. 3B—each (group) remains linearly arranged. Further, locations, initially arranged along a geodesic curve G (the generalization of a straight line to a curved object) that longitudinally extends along the surface of elongated element 310, remain on the curve G, as coupling element 108, and consequently, tip component 110, are rotated about the longitudinal axis A. (The totality of locations indicated by the points p and p′ lie along the curve G.) In other words, since elongated element 310 (practically) cannot be wound about itself, the locations indicated by the points p cannot be displaced along the circumference of elongated element 310 relative to the locations indicated by the points p′. Due to the affixing of elongated element 310 to tip component 110, the same holds true of similarly positioned locations on the surface of tip component 110 and the surface of shaft 106, such as the locations indicated by points p″ and p′″, respectively, in FIG. 3A.

More specifically, locations on the surface of shaft 106, such as the locations indicated by the points p′″, always point in the direction of the x-axis and perpendicularly to the y-axis. Consequently, locations on the surface of tip component 110, such as the locations indicated by the points p″— and therefore first side-camera 210b and first side-illumination modules 220b—which in FIG. 3A point in the direction of the x-axis and perpendicularly to the y-axis, continue to point perpendicularly to the y-axis, as coupling element 108 is rotated about the z-axis.

Intuitively, elongated element 310 can be thought of as inducing a counter rotation of tip component 110 about the longitudinal axis B, such that if tip component 110 is rotated by n degrees in a first sense (e.g. clockwise) about the longitudinal axis A, the rotation is accompanied by a simultaneous rotation of tip component 110 of n degrees in a second and opposite sense (e.g. counter-clockwise) about the longitudinal axis B. In this way, side-cameras 210b and 210c, and side-illumination modules 220b and 220c, continue to point perpendicularly, or substantially perpendicularly, to the y-axis as coupling element 108 is rotated about the longitudinal axis A and endoscope 100 is switched between configurations. In embodiments wherein a direction of the y-axis is visually or tactually indicated on handle 104 (for example, by buttons of user control interface 140 pointing in the direction of they-axis), a user wielding handle 104, e.g. during a surgical procedure, is thus provided with information regarding the orientations of side-cameras 210b and 210c even when elongated member 102 is inserted inside an anatomical cavity of a patient.

FIG. 7 presents a block diagram relating functionalities of some parts and components of endoscope 100, according to some embodiments. Depicted are handle 104, shaft 106, coupling element 108, and tip component 110. Further depicted are a motor 710 (e.g. an electric or an electromagnetic motor) and mechanical infrastructure 720 mechanically associated therewith, as described below. According to some embodiments, motor 710 is housed within handle 104. Mechanical infrastructure 720 may extend from motor 710, via shaft 106, onto coupling element 108. Motor 710 is mechanically coupled to coupling element 108 via mechanical infrastructure 720, such as to allow for a controllable rotation of coupling element 108 about the longitudinal axis A.

According to some embodiments, mechanical infrastructure 720 may be in the form of one or more elongated rods, which motor 710 may be configured to controllably rotate about the longitudinal axis A. The one or more rods may be affixed to inner walls of proximal arm 614 and/or central part 610 of coupling element 108, so that the rotation of the one or more rods about the longitudinal axis A induces a like rotation of coupling element 108. In such embodiments, each rod may be affixed to the respective inner wall of proximal arm 614 or central part 610 via a rung, which extends transversely from the distal end of the rod (and away from the longitudinal axis A) to the inner wall. According to some embodiments, instead of including rods, mechanical infrastructure 720 may include an elongated tube (which extends through shaft 106). The tube may include a flange extending around the distal end of the tube, which affixes the tube to coupling element 108. Alternatively, the affixing may be implemented via one or more rungs.

According to some alternative embodiments, the rotation mechanism may be based on the conversion of longitudinal displacement into a commensurate rotation, e.g. in a similar manner to a crankshaft mechanism. More specifically, according to some such embodiments, motor 710 may be configured to controllably longitudinally translate a rod, which extends through shaft 106 and constitutes a part of mechanical infrastructure 720. In such embodiments, mechanical infrastructure 720 further includes components, which are mechanically coupled to coupling element 108 and which are configured to convert the longitudinal translation into a corresponding rotation of coupling element 108.

User control interface 140 is functionally associated with motor 710, such as to allow a user a user to switch endoscope 100 between configurations.

While in FIG. 3B the mechanism for maintaining the side-cameras perpendicular, or substantially perpendicular, to the y-axis is provided by a torsion-resistant elongated element (i.e. elongated element 310), other options are possible and fall within the scope of the present disclosure. In particular, according to an aspect of some embodiments, there is provided an endoscope, which is essentially similar to endoscope 100 but differs therefrom in including an alternative mechanism for maintaining the side-cameras perpendicular, or substantially perpendicular, to the y-axis. The alternative mechanism does not rely on torsion-resistance. Instead, the mechanism is gear-based. The endoscope thus need not include a torsion-resistant elongated element.

According to some embodiments, the endoscope includes a shaft, a coupling element, and a tip component. The shaft is essentially similar to shaft 106. The tip component is similar to tip component 110 but may differ therefrom as specified below. The coupling element is similar to coupling element 108 but may differ therefrom in including a first gear (e.g. a first cogwheel) and a second gear (e.g. a second cogwheel). The first gear may be affixed to inner walls of the coupling element (e.g. to inner walls of a central part of the coupling element), such that when the coupling element is rotated about a longitudinal axis of the shaft, so is the first gear. The second gear may be housed in a distal arm of the coupling element and is mechanically coupled to the tip component. The mechanical coupling of the second gear to the tip component is such that when the tip component is rotated about a longitudinal axis thereof, so is the second gear and vice-versa. The mechanical coupling there between may be implemented, for example, by means of a rod extending distally, along the longitudinal axis of the tip component, from the second gear to the tip component. The rod is affixed, e.g. on a distal end portion thereof, to the tip component.

The first gear may be positioned on a distal end of the central part and the second gear may be positioned on a proximal end of the distal arm, such as to allow for direct mechanical coupling there between. The mechanical coupling is such that a rotation of one gear induces an opposite-sense rotation of the other gear of the same magnitude. That is, a d degrees clockwise rotation of the first gear induces a d degrees counter-clockwise rotation of the second gear and vice-versa. Thus, rotation of the coupling element about the longitudinal axis of the shaft—in addition to inducing a like rotation of the tip component (i.e. about the longitudinal axis of the shaft)—further induces an opposite sense rotation of the same magnitude of the tip component about the longitudinal axis thereof. In this way, the side-cameras are maintained pointing perpendicularly, or substantially perpendicularly, to a vertical axis of the endoscope. (The vertical axis is perpendicular to the longitudinal axis of the shaft and defines, together therewith, a vertical plane bisecting the endoscope.)

According to an aspect of some embodiments, there is provided an endoscope, which is essentially similar to endoscope 100 but differs therefrom in providing an alternative mechanism for maintaining the side-cameras perpendicular, or substantially perpendicular, to the y-axis. The alternative mechanism does not rely on torsion-resistance. Instead, the endoscope includes an active mechanism for maintaining the side-cameras perpendicular, or substantially perpendicular, to the y-axis, in the sense of including a pair of motors: A first motor is configured for rotating a coupling element of the endoscope about a longitudinal axis of a shaft of the endoscope. A second motor is configured for rotating a tip component of the endoscope about a longitudinal axis of the tip component. The motors may be synchronized, such that when the first motor induces a rotation of the coupling element, and therefore the tip component, of d degrees about the longitudinal axis of the shaft, the second motor induces a rotation of d degrees in the opposite sense (or, what amounts to the same thing, −d degrees in the same sense) about the longitudinal axis of the tip component. The opposite sense rotation about the longitudinal axis of the tip component compensates for the rotation about the longitudinal axis of the shaft, with the result that the side-cameras are maintained perpendicular, or substantially perpendicular, to the y-axis.

According to an aspect of some embodiments, there is provided an endoscope, which is essentially similar to endoscope 100 but differs therefrom in the structure of a coupling element thereof and the mechanical association between the coupling element and a shaft and a tip component of the endoscope. Both the shaft and the tip component may be essentially similar to shaft 106 and tip component 110 of endoscope 100. In particular, the endoscope includes an elongated element, which is essentially similar to elongated element 310 of endoscope 100 and is essentially similarly disposed within the endoscope. The coupling element includes a central part, a proximal arm, and a distal arm. The proximal arm is mechanically coupled to the central part on a central part proximal end portion, such as to allow rotation of the proximal arm relative to the central part about a longitudinal axis of the shaft. Similarly, the distal arm is mechanically coupled to the central part on a central part distal end portion, such as to allow rotation of the distal arm relative to the central part about a longitudinal axis of the tip component.

The proximal arm is further connected (e.g. by screwing mechanism, gluing, fusing, and/or soldering), on a proximal arm external end portion, to a distal end of the shaft. In particular, the proximal arm may form an extension of the shaft, extending therefrom in the distal direction and centered about the longitudinal axis of the of shaft. Similarly, the distal arm is further connected (e.g. by screwing, gluing, fusing, and/or soldering), on a distal arm external end portion, to the proximal end of the tip component. In particular, the distal arm may form an extension of the tip component, extending therefrom in the proximal direction and centered about the longitudinal axis of the tip component. The proximal arm's mechanical coupling to the central part prevents axial, relative motion there between (but allows the rotation of the central part about the longitudinal axis of the shaft). Similarly, the distal arm's mechanical coupling to the central part prevents axial, relative motion there between (but allows rotation of the distal arm about the longitudinal axis of the tip component).

It is noted that the functionality of the endoscope is identical to that of endoscope 100 in the sense that side-cameras on the tip component remain perpendicular, or substantially perpendicular, to the y-axis as the endoscope is switched between configurations.

Referring again to endoscope 100, while elongated element 310 is disposed within endoscope 100, and, in particular, extends through coupling element 108, the scope of the disclosure also covers the case of an endoscope including a coupling element, which may be similar to coupling element 108, but which, in contrast, is disposed within a hollow elongated element. The elongated element is connected on a proximal end thereof to a shaft of the endoscope, which may be similar to shaft 106, and on a distal end thereof to a (distal) tip component of the endoscope, which may be similar to tip component 110. According to some embodiments, the elongated element's connections to the shaft and the tip component may be fluidly sealed (e.g. through soldering, gluing, screwing, and/or the like), such as to prevent debris and fluids from entering into the endoscope during use, and allow the endoscope to withstand autoclave sterilization. Similarly to elongated element 310, the elongated element may be highly torsion-resistant and configured to provide the same functionality to the endoscope. That is, to keep side-cameras on the tip component pointing perpendicularly, or substantially perpendicularly, to they-axis (or, in other words, in parallel, or substantially in parallel, to the zx-plane) as the endoscope is switched between configurations. According to some embodiments, the elongated element may be a hollow and flexible bellows, which may be similar to the bellows described below in the description of FIGS. 8A-10 or FIG. 11.

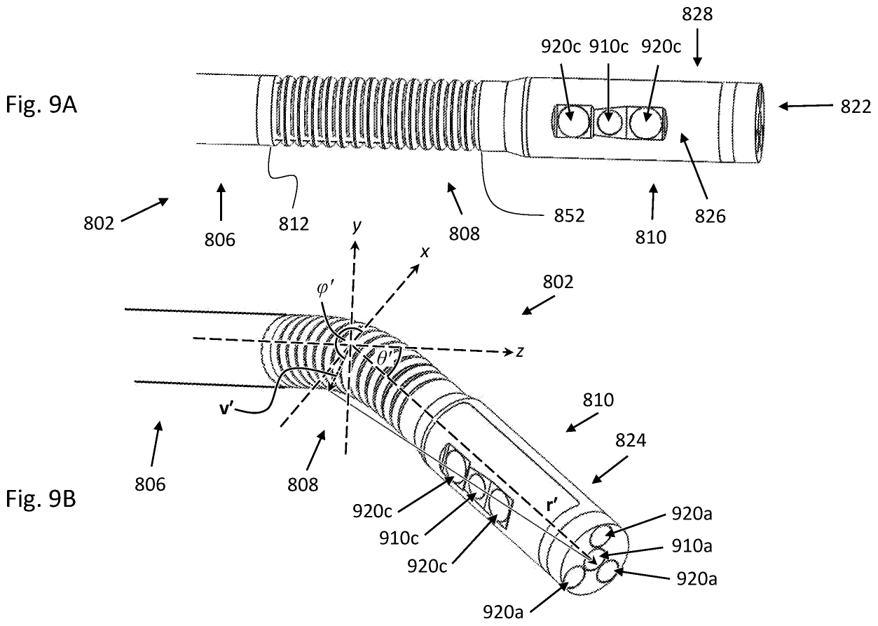

According to an aspect of some embodiments, and as schematically depicted in FIGS. 8A and 8B, there is provided an endoscope 800. According to some embodiments, endoscope 800 includes an elongated member 802, configured to be inserted into an anatomical site, and a handle 804, configured to be held by a user of endoscope 800 and to facilitate guiding and manipulation of elongated member 802 within the anatomical site. Elongated member 802 includes a shaft 806, a neck element 808 (also referred to as “linking element”), and a (distal) tip component 810. Neck element 808 may be connected on a proximal end 811 thereof to a shaft distal end 812 and on a distal end 813 thereof to a proximal end 852 (indicated in FIG. 9A) of tip component 810. According to some embodiments, neck element 808 connections to shaft distal end 812 and to proximal end 852 may be fluidly sealed (e.g. through soldering, gluing, screwing, and/or the like), such as to prevent debris and fluids from entering into the endoscope during use, and allow endoscope 800 to withstand autoclave sterilization.

According to some embodiments, the length of neck element 808 may measure between about 1 millimeter and about 30 millimeters.

Neck element 808 is highly torsion-resistant but may nevertheless be flexible and bendable. More specifically, neck element 808 may be positioned at an adjustable inclination angle θ′ between shaft 806 and tip component 810. More precisely, the angle is spanned between a longitudinal axis L (indicated in FIG. 8B) of shaft 806 and a longitudinal axis M (indicated in FIG. 8B) of tip component 810. The longitudinal axis L centrally extends along the length of shaft 806. The longitudinal axis M centrally extends along the length of tip component 810. According to some embodiments, the inclination angle θ′ may be switchable across a range of angles from about 0 degrees to about 40 degrees, from about 0 degrees to about 30 degrees, or from about 0 degrees to about 22 degrees. Each possibility corresponds to separate embodiments. According to some embodiments, the inclination angle θ′ may be switchable across a range of angles from about 10 degrees to about 30 degrees, from about 15 degrees to about 30 degrees, or from about 15 degrees to about 22 degrees. Each possibility corresponds to separate embodiments.

Also indicated is a user control interface 840, which may be similar to user control interface 140 of endoscope 100. User control interface 840 allows a user to operate endoscope 800, essentially as described above with respect to user control interface 140 of endoscope 100.

Further indicated is a utility cable 850, which may be similar to utility cable 150 of endoscope 100. Utility cable 850 may be configured to functionally associate endoscope 800 with a main control unit, essentially as described above with respect to user control interface 140 of endoscope 100, and/or provide electricity to power endoscope 800 operation. According to some alternative embodiments (not depicted in FIGS. 8A and 8B), endoscope 800 may be functionally associated with the main control unit through wireless communication.

According to some embodiments, endoscope 800 dimensions (discounting neck element 808) may be similar to those of endoscope 100.

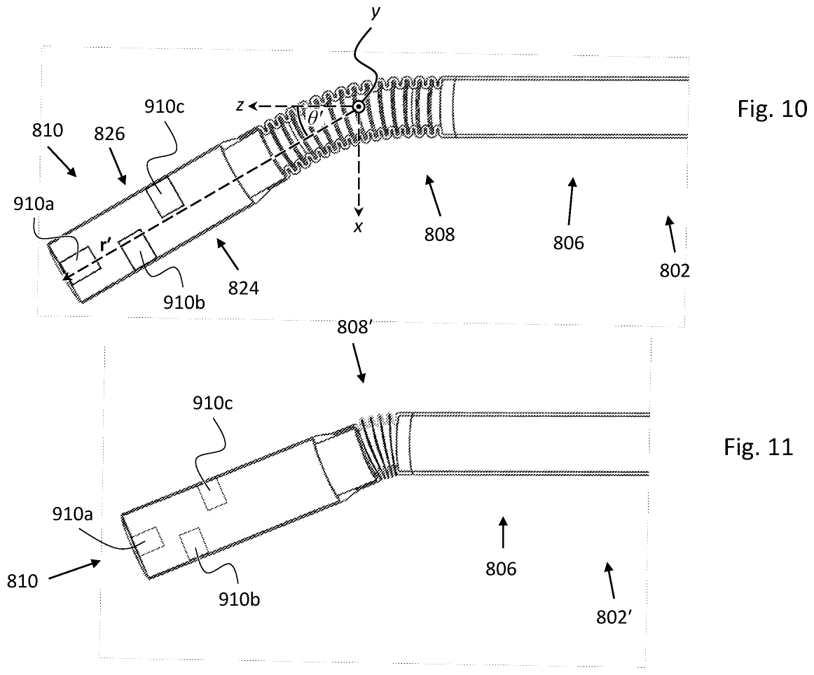

Tip component 110 includes a front surface 822, a first side-surface 824 (not visible in FIGS. 8A and 9A; indicated in FIGS. 9B and 10), and a second side-surface 826, positioned oppositely to first side-surface 824. Also indicated is a top surface 828 of tip component 810. Top surface 828 extends between first side-surface 824 and second side-surface 826.

Tip component 810 may include a plurality of cameras: a front camera and at least one side-camera. Referring also to FIGS. 9A-10, according to some embodiments, tip component includes three cameras: a front camera 910a, a first side-camera 910b (shown in FIG. 10), and a second side-camera 910c, which may be similar to front camera 210a, first side-camera 210b, and second side-camera 210c, respectively, of endoscope 100. Cameras 910 may be positioned on tip component 810 in a similar manner to cameras 210 positioning on tip component 110 of endoscope 100. In particular, cameras 910 may provide similar FOVs to those of cameras 210, respectively. More specifically, cameras 910 may provide a similar combined FOV to that of cameras 210.

Tip component 810 may further includes illumination modules 920 associated with cameras 910. Illumination modules 920 may include: (i) one or more front illumination modules 920a, which are configured to illuminate the FOV of front camera 910a, (ii) one or more first side-illumination modules (not visible in the figures), which are configured to illuminate the FOV of first side-camera 910b, and (iii) one or more second side-illumination modules 920c, which are configured to illuminate the FOV of second side-camera 910c.

To facilitate the description, a cartesian coordinate system is introduced, such that the longitudinal axis L of shaft 806 coincides with the z-axis. The coordinate system is shown superimposed on endoscope 800 in FIGS. 9B and 10. Neck element 808 is positioned at the origin of the coordinate system. The position of a center-point of front surface 822 is given by a vector r′=x\hat{x}+yŷ+z\hat{z}=r\hat{r}, wherein r′ is the magnitude of r′. The longitudinal axis M of tip component 110 thus coincides with the vector r′ and the angle between the vector r′ and the z-axis is given by the inclination angle θ′. In terms of spherical coordinates, the vector r′ is defined by the three coordinates (r′, θ′, φ′). Here θ′ is the polar angle (the spherical coordinates are selected such that the polar angle coincides with the inclination angle defined above). φ′ is the azimuthal angle, which is defined as the angle between the x-axis and the projection of the vector r′ on the xy-plane.

According to some embodiments, endoscope 800 is switchable between configurations, each configuration being characterized by a respective value of the inclination angle θ′ and a respective value of the azimuthal angle φ′. The switching between configurations may be implemented by one or more electric or electromagnetic motors (not shown), and associated mechanical infrastructure (not shown), which are housed in elongated member 802. For example, a first motor may be configured to controllably rotate tip component 810 about the longitudinal axis L and a second motor may be configured to controllably modify the inclination angle θ′.

According to some embodiments, the first motor may be similar, or essentially similar, any one of the motors described in the description of FIG. 7. According to some embodiments, the second motor's working principle may be based on the super-elastic effect. In particular, the second motor may include a memory alloy wire (e.g. a nitinol wire). The second motor may further include electrical infrastructure for passing a current through the wire, such as to heat the wire and thereby induce it to revert back to its “remembered” shape. For example, if the remembered shape is straight, by passing electricity through the wire (when bent), the wire will straighten. According to some embodiments, a spring-mechanism may be used to keep the endoscope 800 at a given value of the inclination angle θ′. When the wire is heated, the wire straightens and counteracts the spring-mechanism such as to straighten endoscope 800.

Endoscope 800 is configured such that side-cameras 910b and 910c face perpendicularly, or substantially perpendicularly, to they-axis in each of the configurations between which endoscope 800 is switchable. More specifically, due to the torsion-resistance of neck element 808, side-cameras 910b and 910c always remain facing perpendicularly, or substantially perpendicularly, to the y-axis, as tip component 810 is rotated about the longitudinal axis L of shaft 806, and, more generally, as the inclination angle θ′ is changed.

According to some embodiments, neck element 808 is a bellows. According to some embodiments, for example, embodiments wherein endoscope 800 is reusable, the bellows may be metallic. More specifically, the bellows may be made of, or include, stainless steel and/or another material, configured to withstand autoclave sterilization (thereby allowing endoscope 800 to withstand autoclave sterilization). According to some alternative embodiments, wherein endoscope 800 is a single-use disposable device, or wherein elongated member 802 is detachably mounted on handle 804 and configured for single-use, the bellows may be made of any material adapted for use in invasive medical procedures, such as a suitable polymeric, ceramic, and/or metallic material, which may be formed into the shape of a bellows with corresponding mechanical properties.

As used herein, the term “bellows” may be used to refer not only to metal bellows, but also to non-metallic elements, which are shaped as a metal bellows and configured to function as a metal bellows in the sense of having similar mechanical properties thereto, high torsion-resistance, as well as flexibility.

Neck element 808 may be hollow or include a passage therethrough for: (i) signal, electricity, and/or power transferring means/elements, such as, but not limited, to cables, electrical wires, optical fibers, and/or printed circuit boards (PCBs; which may be flexible, rigid, flex-rigid, or any combination thereof), which are associated with electronic components in tip component 810 (e.g. the cameras and/or illumination modules); and/or (ii) the motor components and/or mechanical infrastructure (not shown), which are related to the switching between configurations.

FIG. 8A schematically depicts endoscope 800 in a configuration, also depicted in FIG. 9A, wherein the inclination angle θ′ is equal to zero, so that the longitudinal axis L of shaft 806 and the longitudinal axis M of tip component 810 are aligned (i.e. the vector r′ points along the z-axis). First side-camera 910b (hidden from view in both FIG. 8A and FIG. 9A) points in the direction defined by the positive x-axis. Second side-camera 910c (whose optical axis, according to some embodiments, and as depicted in FIGS. 8A and 9A, may be slightly tilted relative to that of first side-camera 910b) may point in a direction, which is parallel to the zx-plane and slightly tilted relative to the direction defined by the negative x-axis.

FIG. 8B schematically depicts endoscope 800 in a configuration, also depicted in FIG. 10, wherein the longitudinal axis M of tip component 810 is positioned at a non-vanishing inclination angle θ′ relative to the longitudinal axis L of shaft 806. More specifically, in terms of the spherical coordinate system of FIGS. 9B and 10, the configuration of FIG. 8B is characterized by an inclination θ′>0° and an azimuthal angle φ′ which is about 270 degrees.

FIG. 9B schematically depicts endoscope 800 in a configuration characterized by an inclination angle θ′>0° and an azimuthal angle 180°<φ<270°. Also indicated in FIG. 9B is a vector v′ indicating the projection of the vector r′ on the xy-plane.

Essentially as described above with respect to handle 104 of endoscope 100, according to some embodiments, handle 804 may include a marking (visual and/or tactile) providing an indication as to directions in which each of side-cameras 910b and 910c, respectively, faces. Additionally, or alternatively, according to some embodiments, user control interface 840 may include one or more buttons 842, which point vertically, i.e. in the direction of the y-axis.

According to some embodiments, endoscope 800 may be (i) directly maneuvered by a user through the manipulation of handle 804, as well as (ii) indirectly maneuvered, via robotics, e.g. using a robotic arm or other suitable gripping means configured to allow manipulation of handle 804.

FIG. 11 presents a schematic view of a distal portion of an elongated member 802′, which is a specific embodiment of elongated member 802, according to some embodiments. Elongated member 802′ includes a shortened neck element 808′, which is a specific embodiment of neck element 808. According to some embodiments, neck element 808′ measures between about 1 millimeters and about 10 millimeters in length. According to some such embodiments, the inclination angle may be between about 0 degrees and about 22 degrees, between about 0 degrees and about 15 degrees, or between about 0 degrees and about 10 degrees. Each possibility corresponds to separate embodiments.

It is noted that the present disclosure applies to reusable endoscopes, which are manufactured to withstand autoclave sterilization (that is, endoscopes which are durable under repeated autoclave sterilization), as well as to single-use disposable endoscopes, which do not need to meet the requirements of autoclave sterilization.

Casings of parts/components of the disclosed reusable endoscopes (e.g. the casing of the shaft, the casing of the handle) may be made of any material, which is resistant to repeated steam autoclaving without loss of dimensional stability and integrity or change in physical characteristics thereof, and which is allowed for use in invasive medical procedures.

According to some embodiments, casings of components of the elongated member may be metallic (e.g. made of stainless steel). According to some embodiments, the casing of the handle may be made of polyphenylsulfone and/or the like.

The sealings of interfaces between different parts/components of the disclosed reusable endoscopes (e.g. the shaft and the handle, the shaft and the coupling element/neck element, the tip component and the coupling element/neck element) are formed not only to keep fluids and debris from entering the endoscope but also to withstand repeated steam autoclaving (thereby allowing for reuse of the endoscope).

Casings of parts/components of the disclosed single-use endoscopes may be made of any material adapted for use in invasive medical procedures (e.g. a suitable polymeric or ceramic material). In contrast to reusable endoscopes, the sealing of interfaces between different parts/components of a single-use endoscope need not be formed to withstand repeated steam autoclaving.

As used herein, according to some embodiments, the term “vertical axis”, with reference to an endoscope (e.g. endoscopes 100 and 800), and the term “y-axis” are used interchangeably.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components set forth herein.

Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1.-31. (canceled)

32. A multi-camera endoscope comprising a handle and an elongated member mounted on the handle and extending distally therefrom, the elongated member comprising:
a proximally positioned shaft;
a distally positioned tip component; and
a linking element affixed on a proximal section thereof to the shaft and on a distal section thereof to the tip component;
wherein the tip component comprises a front camera and two opposite facing, or substantially opposite facing, side-cameras, the cameras being configured to jointly provide a field-of-view (FOV) of at least about 270 degrees;
wherein the endoscope houses at least one motor, and associated mechanical infrastructure, configured for controllable rotation of the tip component about a shaft longitudinal axis, thereby allowing to switch the endoscope between different configurations associated with different FOVs without moving or rotating the handle; and
wherein the linking element is torsion-resistant, being thereby configured to maintain, when rotating the tip component about the shaft longitudinal axis, the side-cameras perpendicular, or substantially perpendicular, to a vertical axis, which is perpendicular to the shaft longitudinal axis and which defines, together therewith, a vertical plane bisecting the endoscope.

33. The multi-camera endoscope of claim 32, wherein a direction defined by the vertical axis is visually and/or tactually indicated on the handle, wherein a user wielding the handle is thereby provided with information regarding orientations of the side-cameras.

34. The multi-camera endoscope of claim 33, wherein the handle comprises a user control interface on a top surface of the handle, the user control interface comprising a plurality of buttons each pointing in a direction of the vertical axis.

35. The multi-camera endoscope of claim 32, further comprising a coupling element comprising a bent central part, a proximal arm, connected to the bent central part, and a distal arm connected to the bent central part, wherein the distal arm is set at a fixed angle relative to the proximal arm;
wherein the proximal arm is mechanically coupled to the shaft and the distal arm is mechanically coupled to the tip component;
wherein (i) the mechanical coupling between the shaft and the proximal arm is configured to prevent displacement of the proximal arm, and therefore the coupling element, relative to the shaft, but to allow for rotation of the coupling element about the shaft longitudinal axis independently of any rotation of the shaft about the shaft longitudinal axis, and (ii) the mechanical coupling between the tip component and the distal arm is configured to prevent displacement of the distal arm, and therefore the coupling element, relative to the tip component, but to allow for rotation of the coupling element about a tip component longitudinal axis independently of any rotation of the tip component about the tip component longitudinal axis, thereby fixing the tip component at the fixed angle relative to the shaft; and
wherein the at least one motor, and associated mechanical infrastructure, are configured to controllably rotate the coupling element about the shaft longitudinal axis, thereby controllably rotating the tip component about the shaft longitudinal axis and switching the endoscope between the configurations.

36. The multi-camera endoscope of claim 35, wherein the mechanical couplings between the shaft and the proximal arm, and between the tip component and the distal arm, additionally provide fluidic-sealing.

37. The multi-camera endoscope of claim 35, wherein the at least one motor, and the associated mechanical infrastructure, are further configured to controllably modify an inclination angle defined between the shaft longitudinal axis and the tip component longitudinal axis.

38. The multi-camera endoscope of claim 37, wherein the inclination angle may be controllably set at any of a plurality of a continuous range of angles between about 0 degrees to about 30 degrees.

39. The multi-camera endoscope of claim 32, wherein the linking element is a bellows connected on a proximal end thereof to a distal end of the shaft and on a distal end thereof to a proximal end of the tip component.

40. The multi-camera endoscope of claim 39, wherein the bellows connection to the shaft and the bellows connection to the tip component are fluidly-sealed.

41. The multi-camera endoscope of claim 35, wherein the coupling element and/or the linking element are configured to withstand autoclave sterilization.

42. The multi-camera endoscope of claim 32, wherein the tip component further comprises a plurality of illumination modules configured to jointly illuminate the FOV of the cameras.

43. The multi-camera endoscope of claim 35, wherein the coupling element and/or the linking element is made of a polymeric, ceramic, or metallic material adapted for use in invasive medical procedures; and
wherein the endoscope is configured for reuse or single-use, or wherein the elongated member is detachably mounted on the handle and is configured for single-use.

44. The multi-camera endoscope of claim 32, further comprising a coupling element comprising a bent central part, a proximal arm, mechanically coupled to the central part, and a distal arm mechanically coupled to the central part, wherein the distal arm is set at a fixed angle relative to the proximal arm;
wherein the proximal arm is connected to the shaft and the distal arm is connected to the tip component, thereby fixing the tip component at the fixed angle relative to the shaft;
wherein the mechanical coupling between the proximal arm and the central part is configured to prevent displacement of the proximal arm, and therefore the shaft, relative to the central part, but to allow for rotation of the central part about the shaft longitudinal axis;
wherein the mechanical coupling between the distal arm and the central part is configured to prevent displacement of the distal arm, and therefore the tip component, relative to the central part, but to allow for rotation of the distal arm and the tip component about a tip component longitudinal axis; and
wherein the at least one motor, and associated mechanical infrastructure, are configured to controllably rotate the central part about the shaft longitudinal axis, thereby controllably rotating the distal arm and the tip component about the shaft longitudinal axis and switching the endoscope between the configurations.

45. The multi-camera endoscope of claim 44, wherein the mechanical couplings between the proximal arm and the central part, and between the distal arm and the central part, additionally provide fluidic-sealing to withstand autoclave sterilization.

46. The multi-camera endoscope of claim 44, wherein the fixed angle ranges between about 10 degrees and about 30 degrees.

47. The multi-camera endoscope of claim 44, wherein the coupling element is hollow and the linking element extends therethrough.

48. The multi-camera endoscope of claim 47, wherein the linking element is a torsion-resistant wire, cable, tube, or bellows.

49. The multi-camera endoscope of claim 44, wherein the linking element is a hollow bellows connected on a proximal end thereof to a distal end of the shaft and on a distal end thereof to a proximal end of the tip component, and wherein the coupling element extends through the bellows.

50. The multi-camera endoscope of claim 49, wherein the coupling element and/or the bellows are made of a material configured to withstand autoclave sterilization, and wherein the bellows connection to the shaft and the bellows connection to the tip component are fluidly-sealed to withstand autoclave sterilization.

51. The multi-camera endoscope of claim 44, wherein the coupling element and/or the linking element is made of a polymeric, ceramic, or metallic material adapted for use in invasive medical procedures; and
wherein the endoscope is configured for reuse, single-use, or wherein the elongated member is detachably mounted on the handle and is configured for single-use.
